(19) United States
(12) Patent Application Publication
(10) Pub. No.: US 2025/0331961 A1
(43) Pub. Date: Oct. 30, 2025

(54) TITANIUM DEVICE FOR GUIDED BONE REGENERATION, AND MANUFACTURING PROCESS

(71) Applicants: Joseph NAMMOUR, PARIS (FR); DEVELOP, Paris (FR)

(72) Inventors: Joseph NAMMOUR, Paris (FR); Georges KHOURY, Paris (FR)

(21) Appl. No.: 18/861,200

(22) PCT Filed: Apr. 25, 2023
(86) PCT No.: PCT/EP2023/060862

§ 371 Date: Oct. 28, 2024

(30) Foreign Application Priority Data
Apr. 28, 2022 (EP) 22305637.5

(51) Int. Cl. A61C 8/02; A61B 6/03; A61F 2/28; A61L 27/06

(52) U.S. Cl. CPC A61C 8/0006; A61B 6/03; A61F 2/2875; A61L 27/06; A61F 2002/2889; A61L 2430/12

(57) ABSTRACT

The present invention relates to a guided bone regeneration device that is intended for the reconstruction of a buccal bone defect and is composed of an optionally micro-perforated smooth wall which is made of titanium and has a shape that covers said buccal bone defect.

The present invention also relates to a process for manufacturing a device of the invention, comprising a step of constructing the device of the invention according to a three-dimensional representation obtained by means of a technique of maxillary-dental imaging of the bone defect.

### CROSS-REFERENCE TO RELATED APPLICATIONS

The present application is a filing under 35 U.S.C. 371 as the National Stage of International Application No. PCT/EP2023/060862, filed Apr. 25, 2023, entitled “TITANIUM DEVICE FOR GUIDED BONE REGENERATION, AND MANUFACTURING PROCESS,” which claims priority to European Application No. 22305637.5 filed with the European Patent Office on Apr. 28, 2022, both of which are incorporated herein by reference in their entirety for all purposes.

### DESCRIPTION

### Technical Field

The present invention relates to a guided bone regeneration device for reconstructing a buccal bone defect, and to a process for manufacturing such a device.

Thus, the present invention finds applications in the medical field, and more particularly the field of dental surgery.

### State of the Art

To regenerate tissue that has been destroyed and/or lost due to bone loss, there are a number of bone augmentation techniques, including apposition grafts, interposition grafts, expansion, distraction, sinus grafts and guided bone regeneration (GBR).

The purpose of GBR is to recreate bone in order to place and/or stabilize a dental implant, or to recreate a ridge for a non-removable prosthesis. Typically, a membrane is placed between the bone and the gum for 3 to 6 months, before the implant can be used or the non-removable prosthesis fabricated. The membrane used forms a physical barrier, preventing colonization of the bone defect by connective and epithelial soft tissues, and limiting access to the scar space to osteogenic cells only. These membranes have a threefold role: to prevent proliferation of cells from the overlying mucosa and to promote migration of cells from the marrow spaces into the site-inhabiting clot, stabilize the bone graft and clot, and stabilize the biomaterial filling.

Several types of membranes, both absorbable and non-absorbable, are used for this purpose, such as polytetrafluoroethylene (PTFE) or absorbable collagen membranes. The main characteristics of non-resorbable membranes are biological inertia, flexibility, chemical stability, and asymmetrical micro-porosity. On the other hand, they require screw fastening and a second operation for removal.

Resorbable membranes, on the other hand, offer the advantage of not having to be removed after bone regeneration, thus avoiding the potential tissue irritation that can occur during removal if the membrane has bonded to the surrounding tissue during the healing phase. However, the disadvantage of resorbable membranes is possible interference between resorption/healing and bone regeneration, as well as the need for a membrane-supporting material.

There is therefore a real need for a device to compensate for these shortcomings, in particular to achieve optimal modeling of the reconstruction, as well as mimicry and symmetry of the bone structures.

### DESCRIPTION OF THE INVENTION

The Applicants have designed a system to meet these objectives.

The device of the invention enables both vertical and horizontal bone augmentation to fill the bone defect, optimal modeling of the reconstruction, and mimicry and symmetry of the bone structures.

Thus, a first object of the invention relates to a guided bone regeneration device that is intended for the reconstruction of a buccal bone defect and is composed of an optionally micro-perforated smooth wall which is made of titanium and has a shape that covers said buccal bone defect.

For the purposes of this invention, “wall” means any non-resorbable wall capable of forming a physical barrier for controlling cell migration during guided bone regeneration (GBR). As the wall is totally smooth, that is, not rough, on both sides of the device, it is totally hermetic, which limits the phenomenon of mucous membranes adhering to the device. In addition, this advantageously implies that the bone will not incorporate the device once it is in place, thus avoiding complications when the device is removed from the mucosa and/or bone.

The wall thickness can be adapted to its function, that is, to protect the biomaterial for the time required for bone reconstruction. In this respect, the person skilled in the art can easily determine the appropriate thickness in each case. For example, the thickness of the device of the invention can be between 0.1 mm and 2.5 mm, for example about 0.1 mm, or 0.2 mm, or 0.3 mm, or 0.4 mm, or 0.5 mm, or 0.6 mm or 0,7 mm or 0.8 mm or 0.9 mm or 1.0 mm or 1.1 mm or 1.2 mm or 1.3 mm or 1.4 mm or 1.5 mm or 1.6 mm or 1.7 mm or 1.8 mm, or 15 1.9 mm, or 2.0 mm, or 2.1 mm, or 2.2 mm, or 2.3 mm, or 2.4 mm, or 2.5 mm.

Advantageously, the device of the invention has a three-dimensional shape covering at least part, and preferably all, of the bone defect. As a result, after bone reconstruction, the reconstructed volume may be substantially the same as that due to treated bone loss, or even greater.

In order to obtain a three-dimensional shape covering the shape of the bone defect to be treated, any technique can be used to model the volume and/or quantity of bone to be reconstructed. This may involve a technique of maxillary-dental imaging of the bone defect, such as cone beam computed tomography. Advantageously, the bone defect can be digitally diagrammed using software suitable for quantifying the bone substance to be regenerated, such as MIMICS or 3-matic software, although this list is not exhaustive.

The device of the invention is thus manufactured according to the modeled shape, so as to mimic 3D planning, by any suitable technique known to the person skilled in the art, such as molding, machining, or 3D printing. Optionally, based on the diagram, an additional 1 mm-thick layer can be added to the 3D representation, before constructing the device of the invention, to advantageously compensate for superficial epithelial formation that can occur in certain regenerations.

Advantageously, the device of the invention has a suitable shape enabling it to cover all or part of the bone defect and allow reconstruction of all the lost bone volume, if necessary. Advantageously, whatever the shape chosen, the device leaves a hollow space between its wall and the residual bone during installation, so as to allow the introduction of a biomaterial inside the device of the invention. Any form that achieves this objective can be used by the person skilled in the art. For example, the shape can be one of a shell, a rigid plate, a hull, or a mesh. However, it cannot be a grid. Regardless of the shape used, the device can be alveolar or non-alveolar (2 mm to 30 mm, round or ovalloid, rectangular or polylobed), to enable the hollow space between the residual bone and the device to be filled with biomaterials or autologous bone.

As previously stated, the device of the invention is made of titanium. The titanium can be pure titanium, or a titanium alloy composed of titanium and at least one other chemical element. The alloys can be any titanium alloy suitable for medical use. In particular, the titanium alloy can comprise at least one other element selected from aluminum, vanadium, iron, hafnium, molybdenum, oxygen, palladium, tin, niobium, zirconium, and tantalum. For example, an alloy selected from Ti-6Al-4V, Ti-6Al-7Nb, Ti-5Al-2.5Fe, Ti-13Ng-13Zr, Ti-12Mo-6Zr-2Fe (also known as “TMZF”), Ti-15Mo-5Zr-3Al, Ti-15Mo-3Nb-3O, Ti-15Zr-4Nb-2Ta-0.2Pd, Ti-15Sn-4Nb-2Ta-0.2Pd, Ti-35Nb-7Zr-5Ta (also known as “TNZT”), Ti-29Nb-13Ta-4.6Zr, Ti-35Nb-5Ta-7Zr-0.4O (also known as “TNZTO”) and Ti—Mo. The titanium content of the device can be between 88% and 100%, for example 88% or 89% or 90% or 91% or 92% or 93% or 94% or 95% or 96% or 100%. This may be a commercial composition, such as Ti Grade 1, 2, 3 or 4. Advantageously, the titanium is used in the device of the invention for its physical properties, such as its resistance to fracture and cracking, its high degree of biocompatibility, its neutrality, and the fact that it induces very little inflammation. Advantageously, the titanium forms a cellular and tissue barrier. What's more, it is biocompatible and non-absorbable, and easy to remove. Thanks to the features of the device of the invention, the graft is advantageously stabilized and compressed, with virtually no resorption.

Advantageously, the device of the invention may comprise at least one perforation for stabilizing the device, each perforation being designed to receive an osteosynthesis screw. The perforation(s) is/are in the walls of the device. The number of perforations can be determined according to conventional criteria applied in this field, for example according to the size of the bone defect. This may involve a number of perforations ranging from 1 to 4, or even 1 to 20 or more, depending on requirements and the volume to be reconstructed.

Advantageously, the device may also comprise microperforations in all or part of its wall. The fact that the device comprises microperforations does not prevent it from being smooth as well, since microperforations do not roughen titanium. It is essential that the device is not rough, to avoid problems during removal due to mucosal or bone adhesion. The microperforations can be distributed uniformly or irregularly over said wall. The microperforations can have a diameter of between 10 μm and 800 μm, for example between 20 μm and 700 μm, or between 50 μm and 600 μm, between 100 μm and 500 μm, or between 150 μm and 300 μm. Advantageously, this range of microperforation diameters makes it possible to achieve cell exclusion between the bone and mucosa compartments, which is not possible with devices, and in particular grids, in the prior art. The density of microperforations on the wall can be between 1 and 100 microperforations per square centimeter, for example between 20 and 90, or between 30 and 80. The holes are usually round, but can be any other shape compatible with their function, for example square, oval, or rectangular. Advantageously, the microperforations can enhance angiogenesis during tissue regeneration. Indeed, the microperforations can allow neovascularization to access the biomaterial from soft tissue, in addition to that arriving via native bone or the native crest to be augmented. The microperforations can also improve soft-tissue adhesion to the titanium plate, avoiding tension in the suture area and reducing initial and late device exposure. The microperforations can be made on the device by any process or tool known to the person skilled in the art, for example using a fine milling cutter or a laser.

Advantageously, the device of the invention can comprise at least one window in its wall, in particular designed for the insertion and condensation of a biomaterial. The biomaterial, once inserted between the bone and the wall of the device of the invention, that is, in the space to be reconstructed, will enable osteoactivity and thus reconstruct the desired bone. Any type of biomaterial suitable for bone reconstruction can be used in this context, as can any type of bone substitute chosen from allogenic, xenogenic, autogenic and synthetic bone substitutes, this list being non-exhaustive. The size, shape, and number of windows can be chosen by the person skilled in the art, depending in particular on the size of the device and the nature of the bone defects to be treated. For example, a window can be square, round, oval, or rectangular. Its dimensions can, for example, be inscribed in a rectangle from 1 to 20 mm high and from 1 to 30 mm wide or more. A window can be designed using any suitable technique known to the person skilled in the art, for example using a 3D planner. The number of windows can be between 1 and 5, for example 1, 2, 3, 4 or 5. However, the number of windows can be adapted by the person skilled in the art to suit their needs. The window(s) can be stabilized by a fixation device, such as an osteosynthesis screw, wires, gears, or interlocking. Advantageously, the function of a window is to enable the filling of the chamber or space created by the device with biomaterials, as well as to enable those biomaterials to settle when the window is supplied and attached. The window has an opening and closing system, so that it can be in the open or closed position. The window can be attached to an opening formed in the wall, through which the biomaterial can be introduced into the chamber or space created by the device, through which the biomaterial can be introduced into the chamber or space inside the device. When the window is in the open position, the opening is exposed and the biomaterial can be inserted into the chamber or space. In the closed position, the window obstructs the opening in order to contain the biomaterial within the device volume and allow it to condense.

Advantageously, the device of the invention is easy to remove once the installation time required for bone reconstruction has been reached. Since it covers the volume to be reconstructed from the outside, it can simply be removed once the screw(s) have been removed.

Advantageously, the device of the invention can also optionally include perforations near the crest, to enable the insertion of dental implants during the surgical stage of guided bone regeneration.

A further object of the invention relates to a process for manufacturing a device of the invention as defined above. In particular, the process implemented may comprise a step of constructing said device as a function of a 3D representation of the bone defect obtained by a maxillary-dental imaging technique, for example by means of a cone beam computed tomography technique.

Advantageously, and as previously indicated, the 3D representation of the bone defect can be digitally diagrammed in suitable software to quantify the bone substance to be regenerated.

If required, the digital diagramming can be used to obtain a diagram, from which the device of the invention will be manufactured so as to cover the 3D representation of the bone defect. If required, an additional 1-mm-thick layer can be added to increase the volume to be covered and obtain a greater reconstructed volume, and to compensate for the formation of a superficial epithelial layer.

Advantageously, the smooth appearance of the device wall can be obtained by any process known to the person skilled in the art. This may involve, for example, a step of 3D printing, machining, or polishing, for example using suitably adapted concave tools.

Another object of the invention relates to a bone regeneration process comprising the steps of:
placing a device according to the invention near a bone defect in a patient,
introducing biomaterial into the device,
keeping the device in place for sufficient time for the biomaterial to condense, and
removing the device of the invention.

Other advantages may be seen by the person skilled in the art by reading the following examples, shown by the appended figures provided by way of illustration.

### BRIEF DESCRIPTION OF THE FIGURES

FIGS. 1A-1C shows a schematic 3-dimensional volumetric bone reconstruction. FIG. 1A: front view of the volume to be reconstructed. FIG. 1B: top view of the volume to be reconstructed. FIG. 1C: bottom view of the volume to be reconstructed.

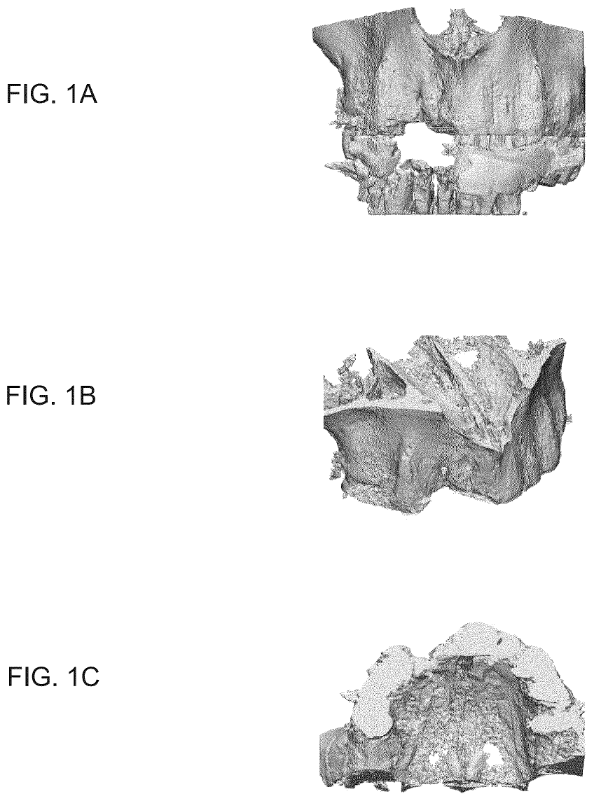

FIGS. 2A-2C shows a view of the bone volume to be reconstructed after cleaning and defining the filling volume using the mirror effect. FIG. 2A: front view of the volume to be reconstructed. FIG. 2B: top view of the volume to be reconstructed. FIG. 2C: bottom view of the volume to be reconstructed.

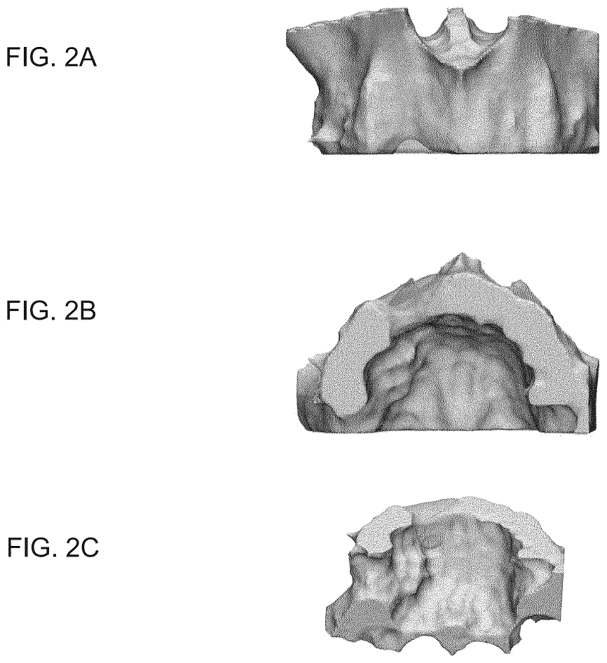

FIGS. 3A-3C shows a view of a 0.8-mm-thick shell with a 1-mm gap above the volume to be filled. FIG. 3A: front view of the volume to be reconstructed. FIG. 3B: top view of the volume to be reconstructed. FIG. 3C: front view of the volume to be reconstructed.

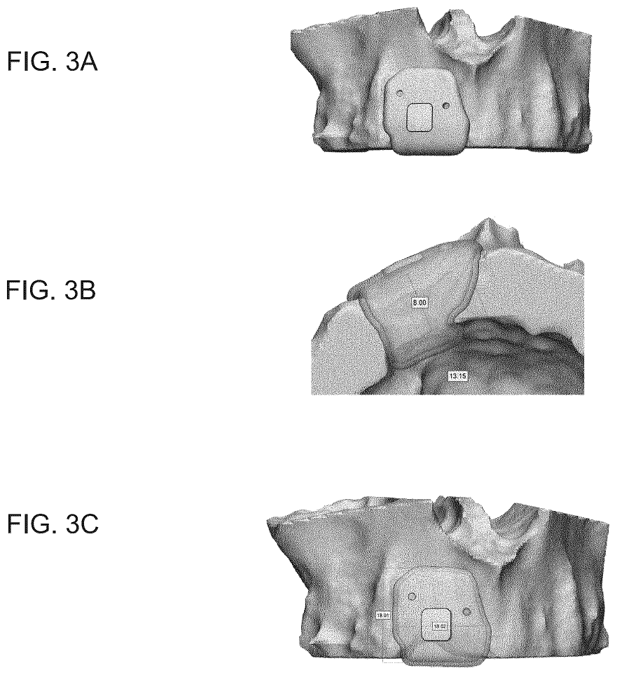

FIG. 4 shows a cross-sectional view of a shell covering the bone volume to be reconstructed.

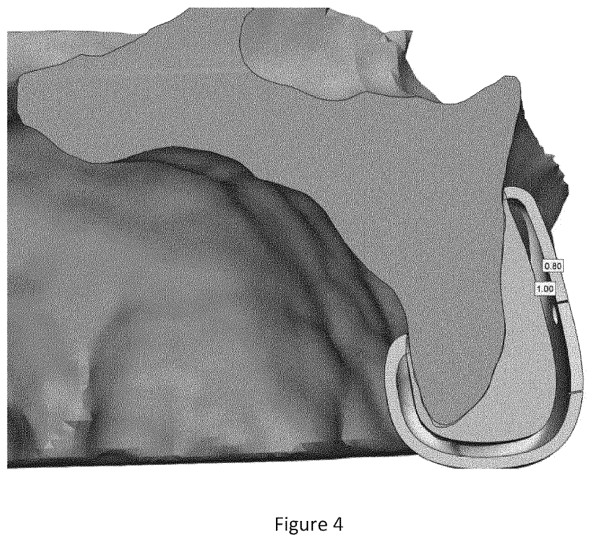

FIG. 5 shows a front view of a shell with a window 6 mm high and 5.5 mm wide, and two fastening holes each 1.4 mm in diameter.

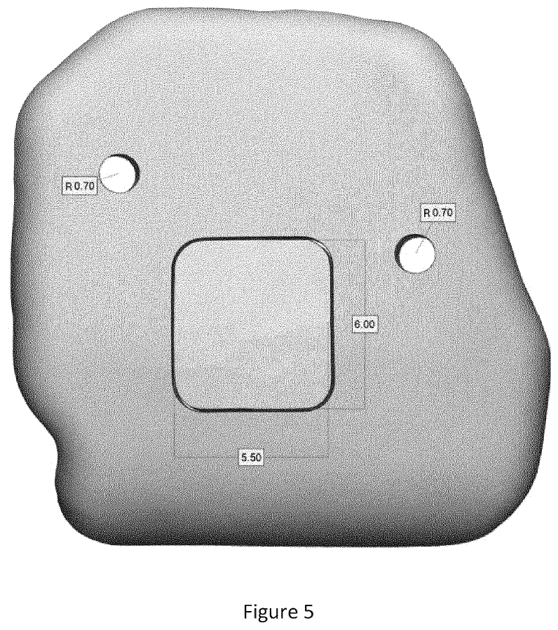

### EXAMPLES

### Example 1: Preparation of a Shell-Shaped Device for Guided Bone Regeneration

The volume of bone to be reconstructed is modeled using cone beam computed tomography.

A 3D representation of the bone defect reconstruction is digitally diagrammed on MIMICS/3-matic software, enabling quantification of the bone substance to be regenerated.

Using this scheme, an additional “optional” 1-mm-thick layer is added to the 3D representation, then a 0.6- to 1.8-mm-thick shell is designed to cover the 3D planning mentioned.

Perforations are drawn to stabilize the future titanium shell with osteosynthesis screws.

Once the shell design has been validated, the shell is printed in titanium using a 3D printer. The device is then polished to a completely smooth surface.

### Example 2: Example Embodiment of the Device for Guided Bone Regeneration

The patient was prescribed preoperative medication: Amoxicillin-clavulanic acid (2 g daily), prednisolone (60 mg daily).

Paracetamol/codeine and mouthwash (0.12 chlorinexidine) were prescribed after the operation.

The procedure was performed under the local anaesthetic Ubistesin® with vasoconstrictor 1/200000.

Decontamination with Betadine® was performed intra- and extra-orally. An incision is made with the 15 C scalpel blade, supra-crestal then intra-sulcular, ending with two buccal release incisions. A full tissue flap was lifted, then the flap released after the periosteal incisions. The flap is dissected using a pair of Metzenbaum® scissors.

The custom titanium mesh/shell is positioned. Drilling was carried out through holes in the existing envelope. The two osteosynthesis screws are partially screwed in, then the allogeneic biomaterials are placed under the shell. The screws were tightened to ensure biomaterial stability. Edge-to-edge sutures were performed without tension.

1. A guided bone regeneration device that is intended for the reconstruction of a buccal bone defect and is composed of an optionally micro-perforated smooth wall which is made of titanium and has a shape that covers said buccal bone defect.

2. The device according to claim 1, wherein the titanium is pure, or is an alloy composed of titanium and at least one other chemical element.

3. The device according to claim 2, wherein said at least one other chemical element is selected from aluminum, vanadium, iron, hafnium, molybdenum, oxygen, palladium, tin, niobium, zirconium and tantalum.

4. The device according to claim 2, wherein said alloy is selected from Ti-6Al-4V, Ti-6Al-7Nb, Ti-5Al-2.5Fe, Ti-13Ng-13Zr, Ti-12Mo-6Zr-2Fe, Ti-15Mo-5Zr-3Al, Ti-15Mo-3Nb-3O, Ti-15Zr-4Nb-2Ta-0.2Pd, Ti-15Sn-4Nb-2Ta-0.2Pd, Ti-35Nb-7Zr-5Ta, Ti-29Nb-13Ta-4.6Zr, Ti-35Nb-5Ta-7Zr-0.4O and Ti—Mo.

5. The device according to claim 1, wherein it has a shape selected from a shell, a plate, and a mesh.

6. The device according to claim 5, wherein said device is alveolar or non-alveolar.

7. The device according to claim 1, comprising at least one perforation for stabilizing said device, said perforation being designed to receive an osteosynthesis screw.

8. The device according to claim 1, wherein the microperforations are arranged on all or part of the wall.

9. The device according to claim 1, comprising at least one window.

10. The device according to claim 9, wherein the window is attached to an opening in the wall.

11. The device according to claim 9, wherein the window has an opening and closing system.

12. The device according to claim 9, wherein said at least one window has dimensions inscribed in a rectangle from 1 to 20 mm high and from 1 to 30 mm wide.

13. The device according to claim 1, with a thickness of between 0.6 mm and 1.8 mm.

14. A process for manufacturing a device as defined in claim 1, comprising a step of constructing said device as a function of a 3D representation obtained by a maxillary-dental imaging technique of the bone defect.

15. The process according to claim 14, wherein said 3D representation of said bone defect is digitally diagrammed on software adapted to quantify the bone substance to be regenerated.

16. The process according to claim 14, wherein said medical imaging technique is a cone-beam computed tomography technique.

17. The process according to claim 14, wherein said digital diagramming makes it possible to obtain a diagram, on which an optional additional layer of 1 mm thickness is added.
